(19) United States
(12) Patent Application Publication
(10) Pub. No.: US 2023/0346398 A1
(43) Pub. Date: Nov. 2, 2023

(54) RE-ENTRY DEVICE FOR VESSEL RECANALIZATION USING A SUBINTIMAL TECHNIQUE

(71) Applicant: C.R. BARD, INC., Franklin Lakes, NJ (US)

(72) Inventors: Amanda Young, Phoenix, AZ (US); Kyle Andrew Thomas, Phoenix, AZ (US); Keith Harris, Mesa, AZ (US); Clint Solomon, Gilbert, AZ (US); Genevieve Messina, Tempe, AZ (US)

(21) Appl. No.: 18/026,685

(22) PCT Filed: Sep. 22, 2020
(86) PCT No.: PCT/US2020/051900

§ 371 Date: Mar. 16, 2023

(51) Int. Cl. A61B 17/22

(52) U.S. Cl. CPC A61B 17/22; A61B 2017/22095; A61B 2017/22038

(57) ABSTRACT

A re-entry device for recanalization of a vessel using a subintimal technique. A catheter (10) includes a first inner lumen (14) extending to a distal end portion (12b) of the catheter, the distal end portion including an angled tip (20) having a distal open end (20a). An elongated stylet (24) is located in the first inner lumen, the stylet having an angled distal end portion (24a) forming a needle. An actuator (26) at a proximal end portion (12a) of the catheter is for advancing the stylet from a retracted position within the catheter to a deployed position projecting from the distal open end of the angled tip for penetrating a wall of the vessel.

### BACKGROUND

When treating peripheral arterial disease, it is common for devices to enter the subintimal space of a vessel wall. The physician must then get back into the true lumen to continue to treat the disease and recanalize the vessel. In some cases, the operator can re-enter without a specialized device, but sometimes arteries are highly calcified or difficult to re-enter using standard techniques.

In larger arteries, such as those located above the knee, several specialized re-entry devices exist to assist the physician in getting out of the arterial wall and reentering the true lumen. However, there are no currently good options for achieving re-entry in small vessels, such as tibial vessels located “below the knee” (BTK). As BTK treatment is increasingly more common, physicians need for a reliable method of re-entry in the tibial arteries.

Accordingly, a need is identified for a re-entry device for recanalization of a vessel using a subintimal technique, and especially one that promotes use in a highly repeatable and thus reliable manner.

### SUMMARY

This disclosure provides a re-entry device for recanalization of a vessel using a subintimal technique, and especially one that is able to be used in a highly repeatable and reliable manner.

According to one aspect of the disclosure, a re-entry device for recanalization of a vessel using a subintimal technique is provided. The device comprises a catheter having a first inner lumen extending to a distal end portion of the catheter. The distal end portion includes an angled tip having a distal open end. An elongated stylet is located in the first inner lumen, the stylet having an angled distal end portion forming a needle. An actuator k provided at a proximal end portion of the catheter for advancing the stylet from a retracted position within the catheter to a deployed position projecting from the distal open end of the angled tip for penetrating a wall of the vessel.

In some embodiments, the catheter includes a second inner lumen for receiving a guidewire. The actuator may be located on a hub of the catheter, and may comprise a slide. The actuator may be manually operable, or may be pneumatic, hydraulic, or magnetic. In one particular version, the actuator comprises a solenoid. The needle may be solid, and the diameter of the catheter may be less than or equal to 4 French (approximately 1.33 millimeters), which thus forms a “micro” catheter. The angled tip may form an acute angle, such as for example about 30 degrees, relative to a longitudinal axis of the catheter.

According to a further aspect of the disclosure, a re-entry device for recanalization of a vessel using a subintimal technique is provided. The device comprises an elongated stylet having an angled distal end portion forming a needle. A catheter includes a first inner lumen in which the elongated stylet is located, a second inner lumen for receiving a guidewire, and an actuator at a proximal end portion of the catheter for advancing the stylet to project the needle from an open distal end of the catheter to facilitate penetration of a wall of the vessel.

In one embodiment, a distal end portion of the catheter comprises an angled tip including the open distal end. The distal end portion of the catheter may be steerable from a control, such as a wire. The actuator may be manually operable, or may be pneumatic, hydraulic, or magnetic. In one particular version, the actuator comprises a solenoid.

A further aspect of the disclosure pertains to a re-entry device for recanalization of a vessel using a subintimal technique. The device comprises an elongated stylet having an angled distal end portion forming a needle. A catheter includes a first inner lumen in which the elongated stylet is located and an actuator at a proximal end portion of the catheter for advancing the stylet to project the needle from an open distal end of an angled tip of the catheter to facilitate penetration of a vessel (arterial) wall. The catheter has a diameter of less than or equal to 1.33 millimeters.

In one embodiment, the catheter includes a second inner lumen for receiving a guidewire. The needle may be solid. The actuator may be manually operable, or may be pneumatic, hydraulic, or magnetic. In one particular version, the actuator comprises a solenoid.

### BRIEF DESCRIPTION OF THE DRAWINGS

The above and further advantages of the disclosure may be better understood by referring to the following description in conjunction with the accompanying drawings in which:

FIG. 1 is a side view of a re-entry device according to a first aspect of the disclosure.

FIG. 1A is a cross-sectional view taken along line 1A-1A of FIG. 1.

FIG. 2 is a side view of a re-entry device of FIG. 1, in an actuated condition.

FIGS. 3-5 are enlarged cutaway views illustrating the re-entry device in a possible mode of use.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the items depicted in the drawings may be combined into a single function.

### DETAILED DESCRIPTION

In the following detailed description, numerous specific details are set forth to provide a thorough understanding of the present invention. The disclosed embodiments may be practiced without these specific details. In other instances, well-known methods, procedures, components, or structures may not have been described in detail so as not to obscure the present invention.

The principles and operation of systems and methods of the disclosure may be better understood with reference to the drawings and accompanying descriptions. The invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

Certain features of the invention that are, for clarity, described in the context of separate embodiments, may also be provided in combination in a single embodiment. Conversely, various features of the invention that are, for brevity, described in the context of a single embodiment, may also be provided separately or in any suitable sub-combination.

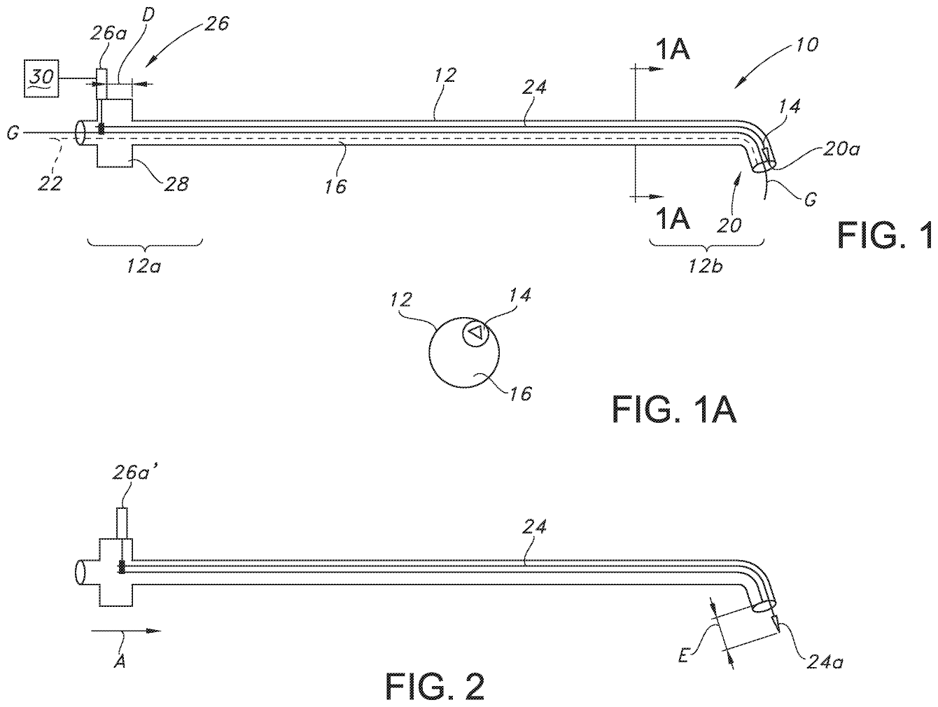

With reference to FIG. 1, a re-entry device 10 for recanalization of a vessel (and, in particular an arterial vessel located “below the knee” (BTK)) using a subintimal technique is proposed. According to one embodiment, the device 10 comprises a catheter 12 having an elongated body, which may be formed of a suitable polymer material via extrusion or molding. The catheter 12 includes a first inner lumen 14 extending from a proximal end portion 12a to a distal end portion 12b of the catheter and a second inner lumen 16 for receiving a guidewire G. The catheter 12 may be considered a “micro” catheter, having a diameter of less than or equal to 4 French (e.g., less than or equal to 1.33 millimeters).

The distal end portion 12b of the catheter 12 includes an angled tip 20. The angled tip 20 includes a distal open end 20a, and may form an acute angle, such as for example 30 degrees, relative to a longitudinal axis of the catheter 12. The angled tip 20 may be substantially rigid and fixed in position, or optionally, may be made sufficiently flexible and steerable using a control (such as a wire 22).

An elongated stylet 24 is located in the first inner lumen 14, and extends from the proximal end portion 12a of the catheter 12 to the distal end portion 12b. The stylet 24 includes a distal end portion 24a forming a needle, which may extend at an angle similar to the angled tip 20 of the catheter 12. The stylet 24 including the distal end portion 24a (needle) may be solid, and may be formed of metal (such as stainless steel).

An actuator 26 is provided at a proximal end portion 12a of the catheter 12. The actuator 26 is adapted to advance the stylet 24 through the first inner lumen 14 from a retracted position within the catheter 12 to a deployed position projecting from the distal open end 20a of the angled tip 20, such as for penetrating an intimal wall of the arterial vessel. The actuator 26 may be located on a hub 28 of the catheter 12 and may comprise a mechanical actuator, such as a slide 26a activatable by a user's digit (such as a thumb) for moving the slide to a forward position (arrow A, 26a′) corresponding to the deployment of the distal end portion 24a of the stylet 24. The distance D of the slide movement is directly proportional to the ejection distance E of the distal end portion 24a, or needle. Alternatively, the actuator 26 may comprise an automatic (pneumatic, hydraulic, or magnetic) structure, including for example a solenoid 30, for advancing and retracting the stylet 24. A spring-loaded actuator 26 may also be used.

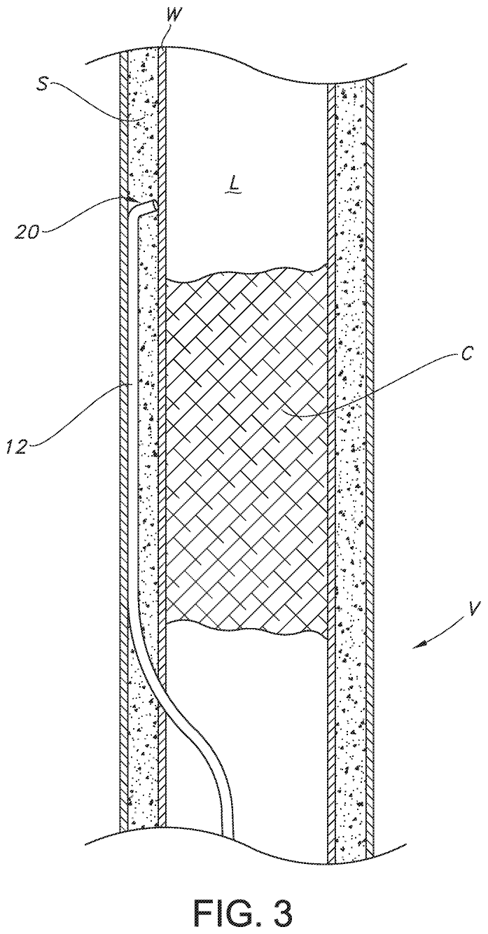

In use, and with reference to FIGS. 3-5 a guidewire (not shown) may be advanced (such as via an anterograde femoral route) along a vessel V towards an occlusion C. The occlusion C may be probed using short advancing movements of the guide wire until resistance is encountered that prevents the correct advancement thereof.

The guidewire is then replaced with a second guide wire (not shown), the advancement of which leads to the bending of the distal floppy end of the guide wire itself, so forming a loop due to the increased flexibility of the distal segment thereof. The loop of the distal end of the second guide wire travels into the thickness of the arterial wall of the occluded vessel, beneath the intima into a subintimal space S. The loop of the second guidewire is advanced up to the level where the lumen L is reconstituted.

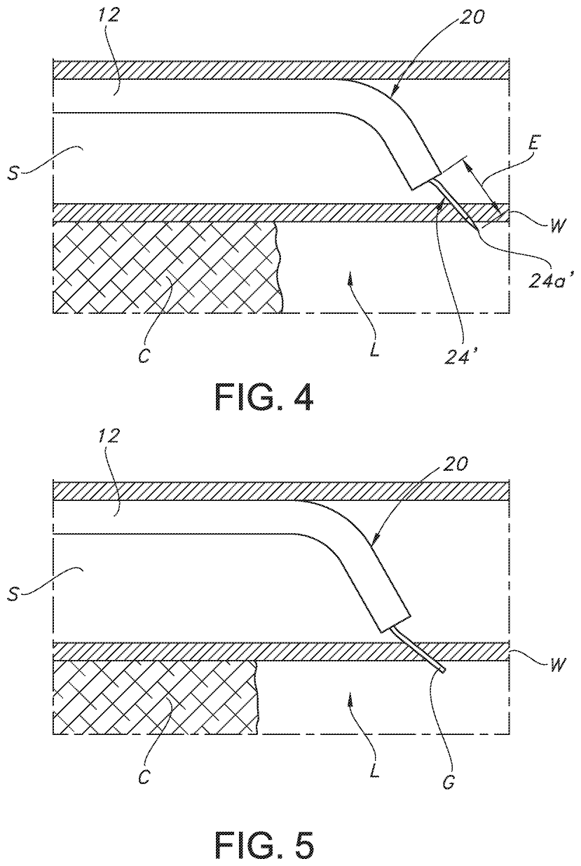

The catheter 12 may then be passed along the second guidewire, which may then be removed. The angled tip portion 20 of catheter 12 is then oriented towards the vessel lumen L, as shown in FIG. 3, with the distal open end 20a facing the arterial wall W to be punctured in order to return into the true lumen L. The stylet 24 is then actuated to exit from the distal opening of catheter 12 (note positions 24,′ 24a′) a distance corresponding to the degree of actuation of the actuator, and thereby reenter the true lumen L, as shown in FIG. 4. As can be appreciated, the deployment or ejection distance E is fixed to ensure that only the wall W is penetrated, and in view of the fixed distance of the actuator 28 is highly repeatable. Furthermore, since the stylet 24 is in position within the catheter 12, and only travels a short distance to eject from the distal open end 20a, there is no need for threading the stylet 24 through the entire catheter 12 to reach the true lumen L.

As shown in FIG. 5, a guidewire G may then be advanced through the distal open end 20a. The stylet 24 may then be retracted, and the catheter 12 removed. A dilatation catheter or other treatment device guided into place along the guidewire G.

Summarizing, this disclosure may be considered to relate to the following items:
1. A re-entry device for recanalization of a vessel using a subintimal technique, comprising:
   a catheter having a first inner lumen extending to a distal end portion of the catheter, the distal end portion including an angled tip having a distal open end;
   an elongated stylet located in the first inner lumen, the stylet having an angled distal end portion forming a needle; and
   an actuator at a proximal end portion of the catheter for advancing the stylet from a retracted position within the catheter to a deployed position projecting from the distal open end of the angled tip for penetrating a wall of the vessel.
2. The device of item 1, wherein the catheter includes a second inner lumen for receiving a guidewire.
3. The device of item 1 or item 2, wherein the actuator is located on a hub of the catheter.
4, The device of any of items 1-3, wherein the actuator comprises a slide.
5. The device of any of items 1-4, wherein the actuator is pneumatic, hydraulic, or magnetic.
6. The device of any of items 1-5, wherein the actuator comprises a solenoid.
7. The device of any of items 1-6, wherein the needle is solid.
8. The device of any of items 1-7, wherein a diameter of the catheter is less than or equal to 4 French.
9. The device of any of items 1-8, wherein a diameter of the catheter is less than or equal to 1.33 millimeters.
10. The device of any of items 1-9, wherein the angled tip forms an acute angle, such as about 30 degrees, relative to a longitudinal axis of the catheter.
11. A re-entry device for recanalization of a vessel using a subintimal technique, comprising:
   an elongated stylet having an angled distal end portion forming a needle; and
   a catheter having a first inner lumen in which the elongated stylet is located, a second inner lumen for receiving a guidewire, and an actuator at a proximal end portion of the catheter for advancing the stylet to project the needle from an open distal end of the catheter to facilitate penetration of a wall of the vessel.
12. The device of item 11, wherein a distal end portion of the catheter comprises an angled tip including the open distal end,
13. The device of item 11 or item 12, wherein a distal end portion of the catheter is steerable from a control,
14. The device of item 13, wherein the control comprises a wire.
15. The device of any of items 11-14, wherein the actuator is pneumatic, hydraulic, or magnetic.
16. The device of any of items 11-15, wherein the actuator comprises a solenoid.
17. A re-entry device for recanalization of a vessel having a wall using a subintimal technique, comprising:
   an elongated stylet having an angled distal end portion forming a needle; and
   a catheter having a first inner lumen in which the elongated stylet is located and an actuator at a proximal end portion of the catheter for advancing the stylet to project the needle from an open distal end of an angled tip of the catheter to facilitate penetration of the vessel wall, the catheter having a diameter of less than or equal to 1.33 millimeters.
18. The device of item 17, wherein the catheter includes a second inner lumen for receiving a guidewire.
19. The device of item 17 or item 18, wherein the needle is solid.
20. The device of any of items 17-19, wherein the actuator is pneumatic, hydraulic, or magnetic.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” or “approximately,” as used herein referring to a measurable value, such as a parameter, an amount, a temporal duration, and the like, is meant to encompass variations of +/−20% or less, including+/−10% or less, +/−5% or less, +/−1% or less, and +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Although the invention has been described in conjunction with specific embodiments, many alternatives, modifications, and variations will be apparent to those skilled in the art. Accordingly, it embraces all such alternatives, modifications, and variations that fall within the appended claims' spirit and scope.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment may also be illustratively described and presented in the context or format of a plurality of separate embodiments.

All publications, patents and patent applications mentioned in this specification are herein incorporated in their entirety by reference into the specification, to the same extent as if each individual publication, patent or patent application was specifically and individually indicated to be incorporated herein by reference. In addition, identification of any reference in this application shall not be construed as an admission that such reference is available as prior art to the present disclosure.

1. A re-entry device for recanalization of a vessel including a wall using a subintimal technique, comprising:
a catheter having a first inner lumen extending to a distal end portion of the catheter, the distal end portion including an angled tip having a distal open end;
an elongated stylet located in the first inner lumen, the stylet having an angled distal end portion forming a needle; and
an actuator at a proximal end portion of the catheter for advancing the stylet from a retracted position within the catheter to a deployed position projecting from the distal open end of the angled tip for penetrating the wall of the vessel.

2. The device of claim 1, wherein the catheter includes a second inner lumen for receiving a guidewire.

3. The device of claim 1, wherein the actuator is located on a hub of the catheter.

4. The device of claim 1, wherein the actuator comprises a slide.

5. The device of claim 1, wherein the actuator is pneumatic, hydraulic, or magnetic.

6. The device of claim 1, wherein the actuator comprises a solenoid.

7. The device of claim 1, wherein the needle is solid.

8. The device of claim 1, wherein a diameter of the catheter is less than or equal to 4 French.

9. The device of claim 1, wherein a diameter of the catheter is less than or equal to 1.33 millimeters.

10. The device of claim 1, wherein the angled tip forms an acute angle of relative to a longitudinal axis of the catheter.

11. A re-entry device for recanalization of a vessel including a wall using a subintimal technique, comprising:
an elongated stylet having an angled distal end portion forming a needle; and
a catheter having a first inner lumen in which the elongated stylet is located, a second inner lumen for receiving a guidewire, and an actuator at a proximal end portion of the catheter for advancing the stylet to project the needle from an open distal end of the catheter to facilitate penetration of the wall of the vessel.

12. The device of claim 11, wherein a distal end portion of the catheter comprises an angled tip including the open distal end.

13. The device of claim 11, wherein a distal end portion of the catheter is steerable from a control.

14. The device of claim 13, wherein the control comprises a wire.

15. The device of claim 11, wherein the actuator is pneumatic, hydraulic, or magnetic.

16. The device of claim 11, wherein the actuator comprises a solenoid.

17. A re-entry device for recanalization of a vessel including a wall using a subintimal technique, comprising:
an elongated stylet having an angled distal end portion forming a needle; and
a catheter having a first inner lumen in which the elongated stylet is located and an actuator at a proximal end portion of the catheter for advancing the stylet to project the needle from an open distal end of an angled tip of the catheter to facilitate penetration of the wall of the vessel, the catheter having a diameter of less than or equal to 1.33 millimeters.

18. The device of claim 17, wherein the catheter includes a second inner lumen for receiving a guidewire.

19. The device of claim 17, wherein the needle is solid.

20. The device of claim 17, wherein the actuator is pneumatic, hydraulic, or magnetic.
